(19) United States
(12) Patent Application Publication
(10) Pub. No.: US 2024/0245340 A1
(43) Pub. Date: Jul. 25, 2024

(54) ELIMINATION OF NEGATIVE COMPONENT OF UNIPOLAR ATRIAL ELECTROGRAM AND SIGNALING OF SAME

(71) Applicant: BioSig Technologies, Inc., Westport, CT (US)

(72) Inventor: Zachary W. KOCH, Cleveland, OH (US)

(73) Assignee: BioSig Technologies, Inc., Westport, CT (US)

(21) Appl. No.: 18/459,715

(22) Filed: Sep. 1, 2023

Related U.S. Application Data
(60) Provisional application No. 63/462,003, filed on Apr. 26, 2023.
(60) Provisional application No. 63/403,500, filed on Sep. 2, 2022.

(51) Int. Cl. A61B 5/339; A61B 5/349

(52) U.S. Cl. CPC A61B 5/339; A61B 5/349

(57) ABSTRACT

Disclosed herein are system and method aspects for monitoring changes in a negative component of a unipolar electrogram. The system can include a memory and at least one processor coupled to the memory. The at least one processor can be configured to receive a unipolar electrogram signal from a catheter, display on at least one digital display a signal waveform based on the unipolar electrogram signal using a first attribute. The signal waveform can represent one or more cycles from a patient's heart. The at least one processor can also be configured to monitor the signal waveform to detect a change in a negative component of the unipolar electrogram signal and to signal the change on the at least one digital display when the change is detected in at least a threshold number of consecutive cycles.

### CROSS-REFERENCES TO RELATED APPLICATIONS

This application is a non-provisional of and claims the benefit and priority to U.S. Provisional Patent Application Nos. 63/403,500, filed Sep. 2, 2022, entitled “Cycle Length Monitoring,” and 63/462,003, filed Apr. 26, 2023, entitled “Elimination of Negative Component of Unipolar Atrial Electrogram and Signaling of Same,” and which are hereby incorporated herein in their entireties by reference.

### BACKGROUND

### Technical Field

Aspects of the present disclosure relate to components, systems, and methods for monitoring negative component changes within cardiac signal data.

### Background

Patients with paroxysmal atrial fibrillation (AF) may be treated with a catheter ablation procedure to achieve pulmonary vein isolation (PVI). During a PVI procedure, a physician may apply radiofrequency (RF) energy to an ablation spot. Unsuccessful treatments and recurrence of AF may be due to the inability to create a transmural lesion. The physician performing a PVI procedure may desire to be able to determine an end point for the RF application.

There is a need to provide an intuitive and easy way for the party performing the PVI procedure to determine when a transmural lesion is created.

### SUMMARY

In aspects presented herein, computing devices can provide for monitoring changes in a negative component of a unipolar electrogram.

In an aspect, disclosed is a system for monitoring changes in a negative component of a unipolar electrogram. The system can include a memory and at least one processor coupled to the memory. The at least one processor can be configured to receive a unipolar electrogram signal from a catheter, display on at least one digital display a signal waveform based on the unipolar electrogram signal using a first attribute. The signal waveform can represent one or more cycles from a patient's heart. The at least one processor can also be configured to monitor the signal waveform to detect a change in a negative component of the unipolar electrogram signal and to signal the change on the at least one digital display when the change is detected in at least a threshold number of consecutive cycles.

In another aspect, a method for monitoring a change in a negative component of a unipolar electrogram signal is described. The method begins by receiving a unipolar electrogram signal from a catheter and displaying, on at least one digital display, a signal waveform based on the unipolar electrogram signal using a first attribute. The signal waveform represents one or more cycles from a patient's heart. The method can monitor the signal waveform to detect a change in a negative component of the unipolar electrogram signal. The method can then signal the change on the at least one digital display when the change is detected in at least a threshold number of consecutive cycles.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIG. 1 is an illustration of a system for cycle length monitoring, according to some aspects of the present disclosure.

FIG. 2 is an example output of the disclosed system, according to some aspects of the present disclosure.

FIG. 3 is a flowchart of a method for monitoring cycle length changes and pattern changes within cardiac signal data, according to some aspects of the present disclosure.

FIG. 4 is a flowchart of a method for monitoring cycle length changes within cardiac signal data, according to some aspects of the present disclosure.

FIG. 5 is a flowchart of a method for monitoring pattern changes within cardiac signal data, according to some aspects of the present disclosure.

FIGS. 6A and 6B show unipolar atrial electrograms before and after radiofrequency (RF) delivery, according to some aspects of the present disclosure.

FIG. 7 is an illustration of a system for monitoring a negative component of a unipolar atrial electrogram, according to some aspects of the present disclosure.

FIGS. 8A and 8B show exemplary outputs of the system for monitoring the negative component of the unipolar atrial electrogram, according to some aspects of the present disclosure.

FIG. 9 is a flowchart of a method for monitoring the negative component of the unipolar atrial electrogram, according to some aspects of the present disclosure.

FIG. 10 is a block diagram of an example computer system useful for implementing various aspects.

In the drawings, like reference numbers generally indicate identical or similar elements. Additionally, generally, the left-most digit(s) of a reference number identifies the drawing in which the reference number first appears.

Aspects of the present disclosure will be described with reference to the accompanying drawings.

### DETAILED DESCRIPTION

It is to be appreciated that the Detailed Description section, and not any other section, is intended to be used to interpret the claims. Other sections can set forth one or more but not all exemplary aspects as contemplated by the inventor(s), and thus, are not intended to limit this disclosure or the appended claims in any way.

While this disclosure describes exemplary aspects for exemplary fields and applications, it should be understood that the disclosure is not limited thereto. Other aspects and modifications thereto are possible, and are within the scope and spirit of this disclosure. For example, and without limiting the generality of this paragraph, aspects are not limited to the software, hardware, firmware, and/or entities illustrated in the figures and/or described herein. Further, aspects (whether or not explicitly described herein) have significant utility to fields and applications beyond the examples described herein.

Aspects have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative aspects can perform functional blocks, steps, operations, methods, etc. using orderings different than those described herein.

References herein to “one aspect,” “an aspect,” “an example aspect,” or similar phrases, indicate that the aspect described can include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it would be within the knowledge of persons skilled in the relevant art(s) to incorporate such feature, structure, or characteristic into other aspects whether or not explicitly mentioned or described herein. Additionally, some aspects can be described using the expression “coupled” and “connected” along with their derivatives. These terms are not necessarily intended as synonyms for each other. For example, some aspects can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary aspects, but should be defined only in accordance with the following claims and their equivalents.

Provided herein are apparatus, device, system, method and/or computer-readable medium aspects, and/or combinations and sub-combinations thereof for monitoring for cycle length and pattern changes within cardiac signal data.

There are two technological problems associated with monitoring cycle length changes and pattern changes within cardiac signal data. First, physicians performing electrophysiology procedures are often inundated with large amounts of data. For example, during an electrophysiology procedure, the display can show signal data associated with one or more catheters. In this scenario, it may be difficult for a physician to detect a cardiac event, such as tachycardia. Second, certain cardiac events can present with characteristics unique to the patient. Physicians can have trouble distinguishing between life threatening and other medical events from the unique characteristics of an individual patient.

Aspects herein solve these technological problems using an innovative method monitoring cycle length changes and pattern changes within cardiac signal data. For example, the disclosed method allows for a display to assign colors to received cardiac signals. Parameters associated with the cardiac signals such as the rate and pattern are compared to templates. Each template can correspond to various cardiac events such as tachycardia or arrhythmia. If the parameters deviate from the template by more than a certain threshold, the color of the signal is changed on the display to alert the physician or other viewing party.

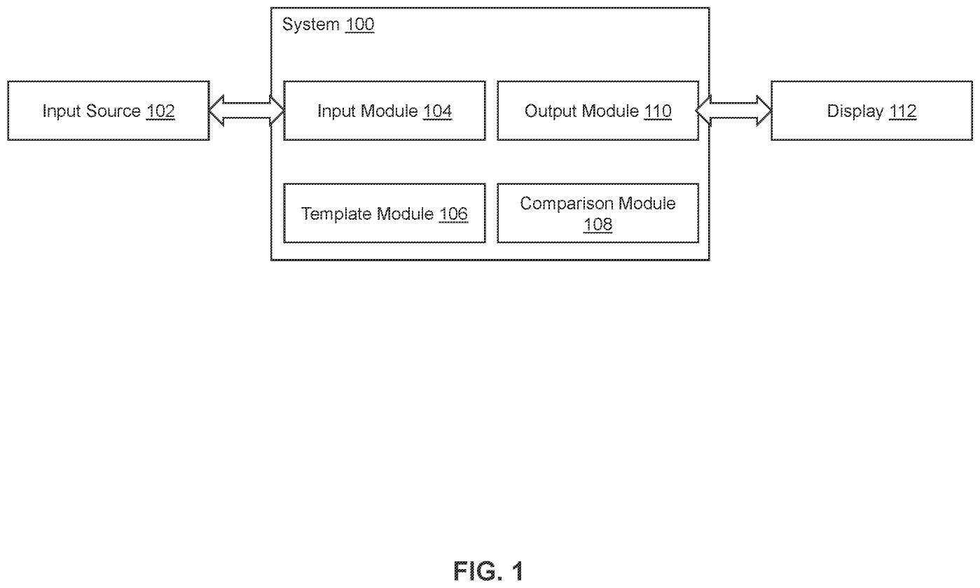

FIG. 1 is a block diagram of a system 100 for monitoring cycle length changes and pattern changes within cardiac signal data. In an embodiment, cycle length changes can correspond to changes in the rate of the heart. Additionally, pattern changes can correspond to changes in the amplitude of the cardiac signal data. System 100 can include an input module 104, a template module 106, a comparison module 108, and an output module 110. System 100 can be in connection with input source 102 and display 112.

Input source 102 can be any source capable of providing catheter based signals of a human or animal heart, for example. In one embodiment, input source 102 can be catheter based signals from intra-cardiac electrogram (EGM) channels. In another embodiment, input source 102 can be a recording containing one or more catheter based signals from intra-cardiac electrogram (EGM) channels. The recording can be stored on a device such as a memory device (e.g., such as a solid state drive, floppy disk drive, a magnetic tape drive, a compact disk drive, an optical storage device, tape backup device, and/or any other storage device/drive). The data provided by input source 102 can contain a rate and/or pattern heart data.

Input module 104 can be any hardware, firmware, and/or software capable of interfacing with input source 102 in order to receive the cardiac signal data from input source 102. In one embodiment, input module 104 can interface with catheter based signals from intra-cardiac EGM channels. In another embodiment, input module 104 can read data from the memory device. Input module 104 can forward the received cardiac signal data to template module 106.

Template module 106 can be any hardware, firmware, and/or software capable of accessing and/or creating one or more templates. Each template can contain one or more parameters. In one embodiment, a template can contain one parameter for a rate and another parameter for a pattern. In another embodiment, a template can contain a single parameter such as a rate. Each parameter can have a corresponding value. For example, a template with a rate parameter can have a corresponding value of 100 beats per minute. A physician (or, e.g., user) can update the value associated with each parameter. Each parameter can also have an associated threshold. In one embodiment, a rate threshold (e.g., a value above or below) can be defined as 10 beats per minute, for example. The threshold can be used to determine the acceptable amount the cardiac signal data can vary from the template parameter. In the above example, cardiac signal data with a rate of 108 beats per minute would be within the template threshold. However, cardiac signal data with a rate of 89 beats per minute would be outside the template threshold. In one embodiment, a physician can update the template threshold.

Additionally or optionally, each template can also have a cycle threshold. A cycle threshold can correspond to the number of cycles that received signal data must differ from the threshold before an alert is triggered. In one embodiment, the cycle threshold can be defined as a number of heart beats. In another embodiment, the cycle threshold can be defined as a time period such as 5 seconds. In some embodiments, no cycle threshold can be specified. Using a cycle threshold can be beneficial to ensure that system 100 does not detect an event that is random or transitory. In one embodiment, a physician can update the cycle threshold.

Template module 106 can also create templates based on data from input source 102. In one embodiment, if input source 102 is a set of catheter based signals, template module 106 can create a rate template based on the detected heart rate of the patient. In the same embodiment, template module 106 can also create a pattern template based on the pattern of the data from the catheter based signals. Each template can also be tagged to correspond to a specific input source 102. For example, if input source 102 is a catheter based signal, one rate template can correspond to a first catheter and a different rate template can correspond to a different catheter. This may be beneficial since each catheter may produce data comprising different values.

Template module 106 can also be capable of saving any templates that are created. In another embodiment, template module 106 can load already created templates from a memory device, such as any of those noted above. Template module 106 can read from and/or write to the memory device.

Comparison module 108 can be any hardware, firmware, and/or software to use the templates from template module 106 to analyze the signal data from input source 102. Comparison module 108 can compare the signal data with the value defined in each template. If the signal data differs from the value defined in each template by more than the threshold in the template, an alert can be triggered. The comparison module 108 can also determine whether the difference between the received value and the template value has occurred for more cycles than the cycle threshold in the template. If no threshold is defined in a template, any difference between the received value and template value can trigger an alert. Additionally, if no cycle threshold is defined in a template, the first difference that occurs can trigger an alert.

Such alerts can contain various pieces of information such as the data from the input source 102 (e.g., 150 beats per minute), the parameter type (e.g., rate, pattern), the parameter value (e.g., 100 beats per minute), the corresponding threshold value (e.g., 10 beats per minute), the cycle threshold, and the number of cycles the input source has differed from the parameter value. Comparison module 108 can send any alerts that are generated to output module 110.

Output module 110 can be responsible for receiving alerts from comparison module 108. Output module 110 can label the signal data corresponding to the received alert. For example, in an embodiment where input source 102 is a catheter based signal and an alert corresponds to one of the catheters, output module 110 can change the color on an intra-cardiac channel display to notify the physician worker of the alert. Output module 110 can forward the alert information a plurality of devices. In one embodiment, output module 110 can send the alert information to a display 112. In another embodiment, output module 110 can forward the alert information to a device in electronic communication with system 100. Output module 110 can communicate and interact over a network with any combination of remote devices, remote networks, remote entities, etc. Each of these devices can be capable of receiving the generated alert. Moreover, in combination with the disclosed embodiments an audible alert can be generated by output module 110 when a given displayed signal's color is changed. Implementation of such an audible alert with hardware, firmware, software, or a combination thereof, would become apparent to persons of skill in the art.

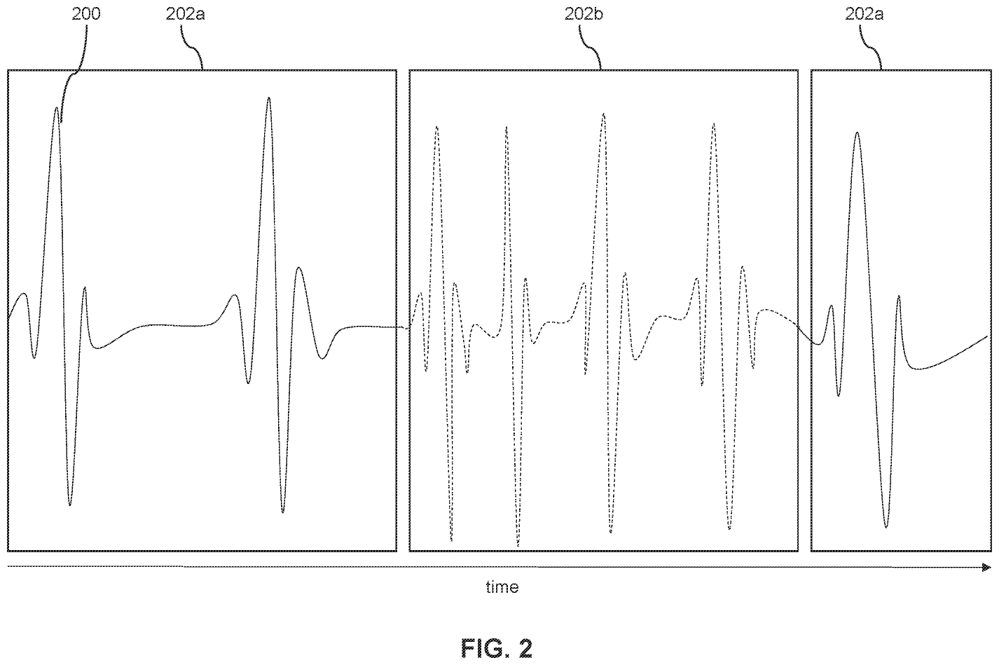

FIG. 2 is an example output of system 100. The display can show intra-cardiac channel data 200. The intra-cardiac channel data 200 can contain a rate and pattern associated with the heartbeat. If the rate and pattern of the intra-cardiac channel data 200 are within the bounds set by the template threshold(s), intra-cardiac channel data 200 will be displayed in a first color 202a (the first color being represented by a solid line for illustration). Conversely, if the rate or pattern change to values outside one or more template thresholds, intra-cardiac channel data 200 can be displayed in a second color 202b (the second color being represented by a dashed line for illustration).

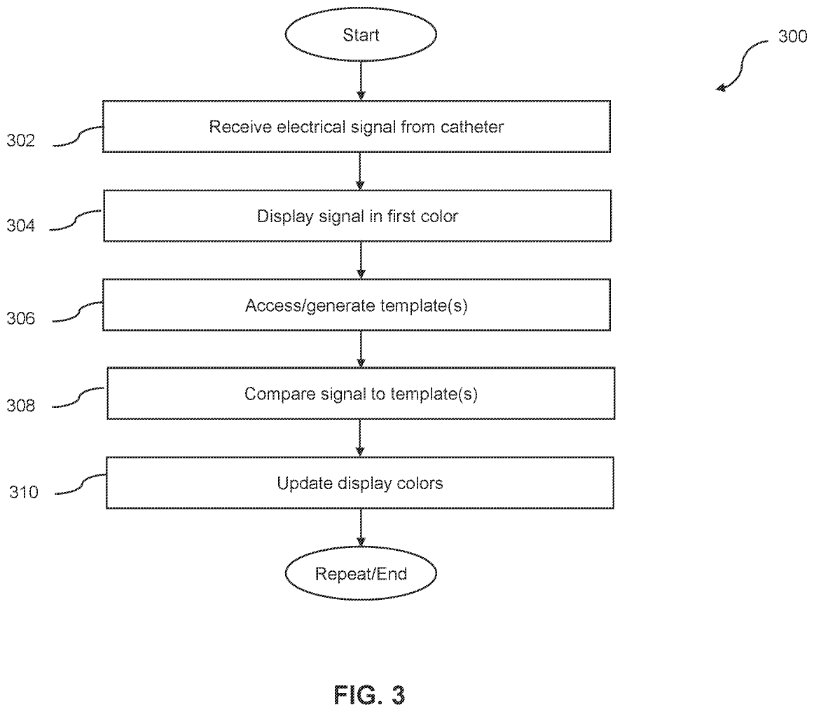

FIG. 3 is a flowchart for a method 300, according to an aspect of the invention.

In 302, an electrical signal is received from a catheter. In one embodiment, the catheter can be used within a system performing an electrophysiology procedure on a patient. The electrical signal can be a cardiac signal originating from a patient's heart.

In 304, the electrical signal is displayed in a first color. The first color (e.g., a default color like green) can be defined and/or updated by a physician, or can be predefined in the system. In one embodiment, the first color signifies that data contained within the electrical signal are within normal bounds.

In 306, one or more templates can be generated. Each template can correspond to a parameter in the electrical signal such as rate or pattern. Each template can have a value specified for the parameter. For example, a template can contain a parameter for a rate and have an associated value of 100 beats per minute. The one or more templates can be generated based on the data within the electrical signal. In another embodiment, the one or more templates can be loaded from a storage device.

In 308, the signal is compared to the one or more templates. The values of the parameters within the signal can be compared to the parameter values of each template. For example, a heart rate within the signal can be compared with each template that has a rate parameter value set.

In 310, the display colors can be updated. If any parameter within the signal differs from a template parameter value by more than a corresponding threshold, the display will be updated to show the signal in a second color (the green signal color is changed to red, for example). The second color can be defined and/or updated by a physician, or can be predefined in the system.

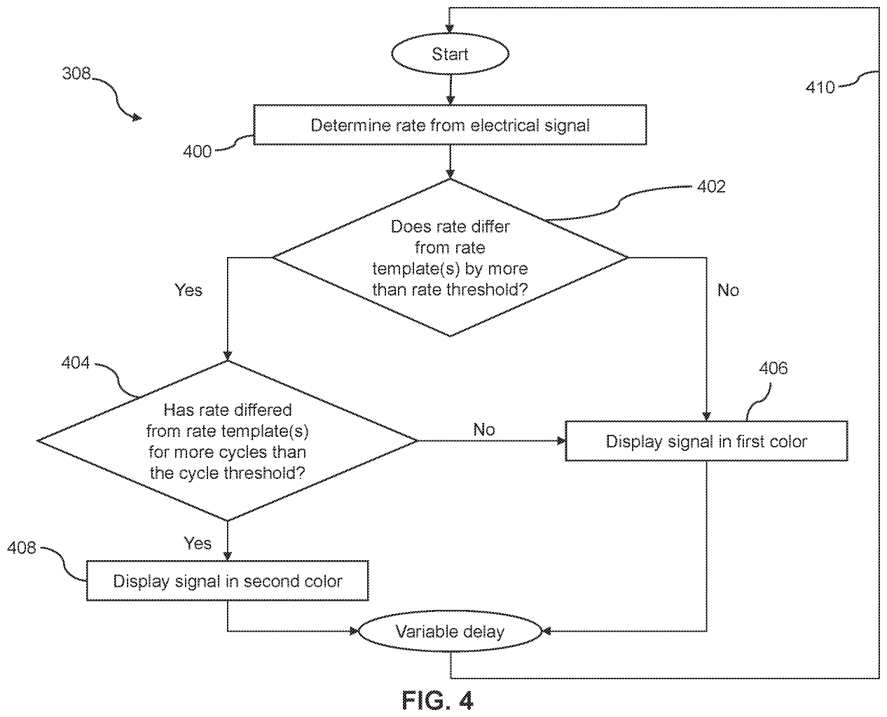

FIG. 4 is a flowchart for method 308 for monitoring cycle length changes and pattern changes within cardiac signal data, according to some aspect of the invention. It is to be appreciated that not all steps can be needed to perform the disclosure provided herein. Further, some of the steps can be performed simultaneously, or in a different order than shown in FIG. 4, as will be understood by a person of ordinary skill in the art.

Method 308 can be implemented and performed by computing device 102. However, method 308 is not limited to that example aspect.

In 400, a rate is determined from an electrical signal. The electrical signal can correspond to the electrical activity of a human or animal heart, for example. The rate can correspond to the speed at which the heart is beating over an amount of time. In one embodiment, the rate can be expressed as beats per minute, but other scales can be used as would become apparent to persons skilled in the art.

In 402, the rate is compared to one or more rate templates. Each rate template can contain a rate value that is of interest. For example, a physician may be interested to know whether a patient is experiencing tachycardia, in which case the rate value of a template can be the beats per minute associated with a tachycardia episode. The template can also have a threshold. The threshold can be used as the acceptable range from which the received value can differ from the template value. If the rate differs from the rate template value by more than the threshold, the method proceeds to step 404.

In 404, the method can determine if the rate differed from the rate threshold for a number of cycles greater than the cycle threshold. The cycle threshold can be a value corresponding to the number of cycles that a received value must differ from the threshold before the color of the displayed signal is changed. This may be beneficial in a scenario in which a patient experiences a brief change in heart rate and/or pattern, but the change is only momentary. In one embodiment, a physician can update the cycle threshold.

In 406, either the rate did not differ from the rate template by more than the rate threshold or the rate did not differ from the rate threshold for more cycles than the cycle threshold. As a result, the signal is displayed with the first color.

In 408, the display will update the color corresponding to the rate to a second color. The second color is used to alert the physician that the patient is or has experienced a heart rate that differs more than the value in the one or more templates, for a number of cycles greater than the cycle threshold. The value of the second color can be updated by the physician.

In 410, a variable delay occurs. The variable delay will pause method 308 for a time according to a predetermined value. In one embodiment, the variable delay will pause method 308 for 10 ms. After the variable delay, method 308 is repeated.

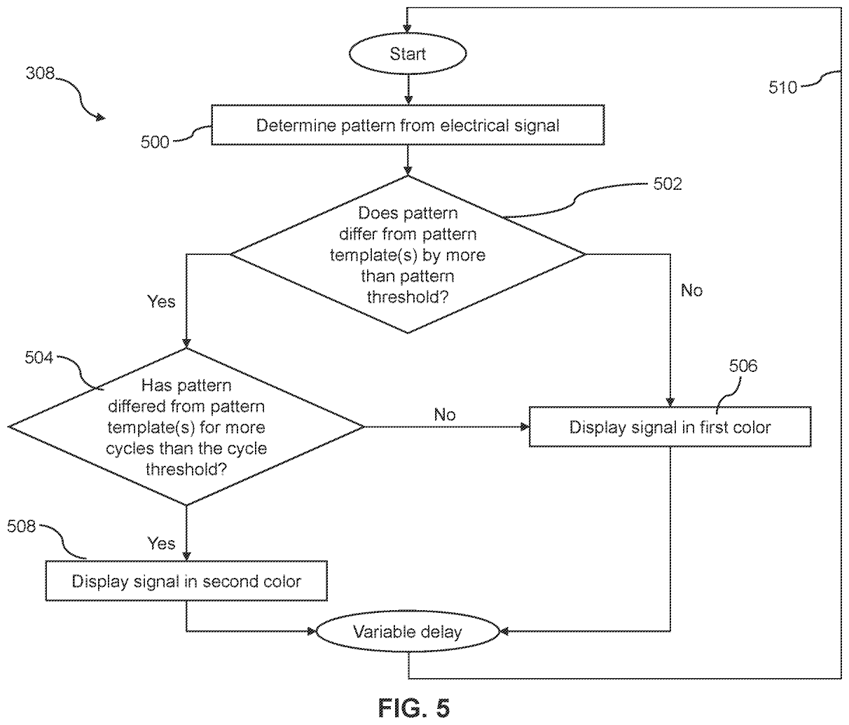

FIG. 5 is a flowchart for method 308 for monitoring cycle length changes and pattern changes within cardiac signal data, according to some aspect of the invention. It is to be appreciated that not all steps can be needed to perform the disclosure provided herein. Further, some of the steps can be performed simultaneously, or in a different order than shown in FIG. 5, as will be understood by a person of ordinary skill in the art.

Method 308 can be implemented and performed by computing device 102. However, method 308 is not limited to that example aspect.

In 500, a pattern is determined from an electrical signal. The electrical signal can correspond to the electrical activity of a heart. The pattern can correspond to the rhythm at which the heart is beating

In 502, the pattern is compared to one or more pattern templates. Each pattern template can contain a pattern value that is of interest. For example, a physician can be interested to know whether a patient is experiencing tachycardia, in which case the pattern value of a template can be the pattern associated with a tachycardia episode. The template can also have a threshold. The threshold can be used as the acceptable range from which the received value can differ from the template value. If the pattern differs from the pattern template value by more than the threshold, the method proceeds to step 504.

In 504, the method will determine if the pattern differed from a pattern threshold for a number of cycles greater than the cycle threshold. The cycle threshold can be a value corresponding to the number of cycles that a received value must differ from the threshold before the color of the displayed signal is changed. This may be beneficial in a scenario in which a patient experiences a brief change in heart rate and/or pattern, but the change is only momentary. In one embodiment, a physician can update the cycle threshold.

In 506, either the pattern did not differ from the pattern template(s) by more than the pattern threshold or the pattern did not differ from the pattern threshold for more cycles than the cycle threshold. As a result, the signal is displayed with the first color.

In 508, the display will update the color corresponding to the pattern to a second color. The second color is used to alert the physician that the patient is or has experienced a cycle pattern that differs more than the value in the one or more templates, for a number of cycles greater than the cycle threshold. The color values can be updated by the physician or set as default colors. In one embodiment, an audible alert can be generated when the display color is updated. Implementation of such an audible alert with hardware, firmware, software, or a combination thereof, would become apparent to persons of skill in the art.

In 510, a variable delay occurs. The variable delay will pause method 308 for a time according to a predetermined value. In one embodiment, the variable delay will pause method 308 for 10 ms. After the variable delay, method 308 is repeated.

Patients with paroxysmal atrial fibrillation (AF) may be treated with a catheter ablation procedure to achieve pulmonary vein isolation (PVI). During a PVI procedure, transmural lesions are created by applying radiofrequency (RF) energy. Unsuccessful treatments and recurrence of AF may be due to the inability to create a transmural lesion. Conventional techniques when performing a PVI procedure do not rely on patient specific metrics to determine when the RF energy application may be stopped. For example, criteria to stop the RF energy application are not related to how the patient is reacting to the RF energy application. The criteria to stop the RF energy application is based on time, power used, and contact power. The physician is not able to detect a response of the tissue and the treatment is not tailored to the patient.

Disclosed herein are methods and systems for monitoring the response from the tissue in real time during RF energy application. The unipolar atrial electrogram (EGM) (also referred to herein as unipolar EGM or EGM) is monitored to detect changes in the electrical behavior of the tissue. Based on the behavior of the tissue, the treatment may be stopped or continued until a desired response is obtained. The unipolar atrial electrogram may be collected using an end of a catheter. Elimination of a negative component of the unipolar atrial electrogram while applying RF energy can be an indication of the formation of the transmural lesion. Thus, the RF energy application may be stopped once the negative component is eliminated.

This provides the advantage of delivering RF energy for the adequate time without the risk of under delivering or over delivering such energy. In some aspects, the transmural lesion may be achieved in 10 seconds when using the elimination of the negative component of the unipolar EGM as an endpoint, compared to around 40 seconds using conventional methods. The excess RF application time does not provide additional benefits to the patient but it may cause harm (e.g., injury to surrounding organs) and wasting RF energy and time.

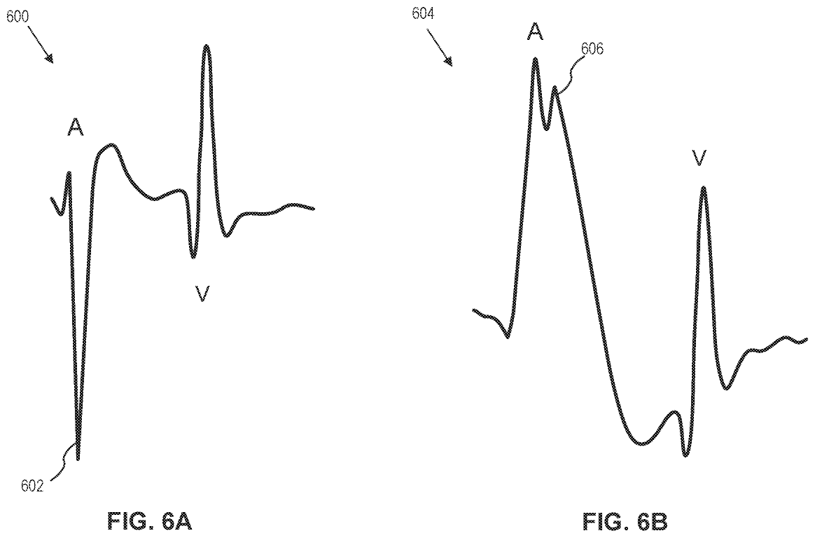

FIG. 6A illustrates a unipolar electrogram before RF delivery, in accordance with some aspects of the present disclosure. EGM 600 shows a positive-negative morphology. The negative component in EGM 600 is indicated by label 602.

FIG. 6B illustrates the unipolar EGM after RF delivery (post ablation), in accordance with some aspects of the present disclosure. EGM 604 shows a positive morphology as indicated by label 606. The negative component of the unipolar EGM is eliminated.

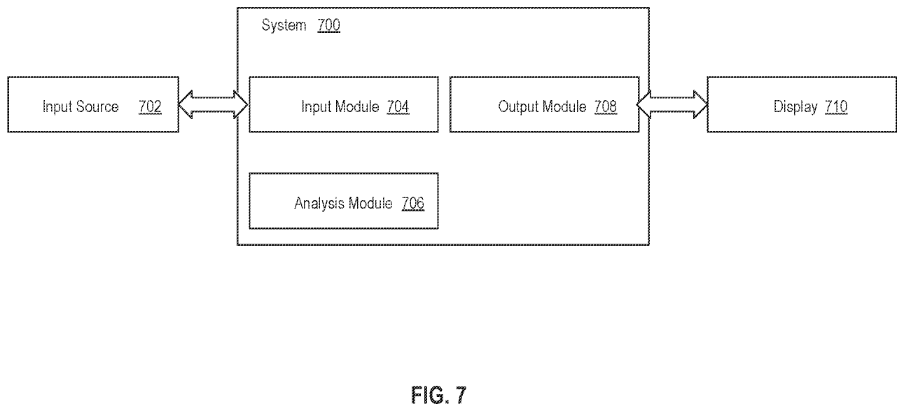

FIG. 7 is a block diagram of a system 700 for detecting unipolar signal modification within cardiac signal data, in accordance with some aspects of the present disclosure. System 700 can include an input module 704, an analysis module 706, and an output module 708. System 700 can be in connection with an input source 702, and a display 710.

Input source 702 can be any source capable of providing catheter based signals of a human or animal heart, for example. In one embodiment, input source 702 can be catheter based signals from unipolar electrogram channels. In another embodiment, input source 702 can be a recording containing one or more catheter based signals from unipolar electrogram channels. The recording can be stored on a device such as a memory device (e.g., a solid state drive, floppy disk drive, a magnetic tape drive, a compact disk drive, an optical storage device, tape backup device, and/or any other storage device/drive). The data provided by input source 702 can represent a clean signal of the unipolar atrial electrogram.

Input module 704 can be any hardware, firmware, software, and/or combinations thereof, capable of interfacing with input source 702 in order to receive the cardiac signal data from input source 702. In one embodiment, input module 704 can interface with catheter based signals from unipolar EGM channels. In another embodiment, input module 704 can read data from the memory device. Input module 704 can forward the received cardiac signal data to analysis module 706.

Analysis module 706 can be any hardware, firmware, software, and/or combinations thereof, capable of analyzing the signal data from input source 702. Analysis module 706 can store one or more parameters. Analysis module 706 can determine and/or store a baseline parameter. The baseline can be identified dynamically based on the cycle received by input source 702. The baseline parameter may be used to determine whether a negative component of the unipolar EGM has been eliminated. In some embodiments, analysis module 706 can determine the baseline based on the detected cycle before the RF energy treatment has begun (e.g., before RF energy application).

Analysis module 706 can also store a cycle threshold. A physician (or e.g., a user) can update a value associated with the cycle threshold. The cycle threshold can be used to determine when to signal to the physician that the RF application may be stopped (i.e., endpoint). The cycle threshold can correspond to the number of cycles that received data do not show the negative component (e.g., the negative component is not detected). In some embodiments, the threshold may have a predetermined value of three.

Analysis module 706 can also store a threshold period. The threshold period may represent a delay period before signaling the elimination of the negative component of the unipolar EGM. The elimination of the negative component is not signaled until the threshold period has elapsed. The threshold period may be 1 second, 2 seconds, 3 seconds, 4 seconds, 5 seconds, or any desired value set by the physician. In some aspects, the physician can update the value associated with the threshold period.

Analysis module 706 can compare the data signal with the baseline to determine whether the cycle comprises the negative component. Analysis module 706 can update a counter value (e.g., increment by one) if the negative component is not detected. Analysis module 706 can output an endpoint notification to output module 708. Analysis module 706 can output the endpoint notification when the negative component is not detected for k-consecutive cycles (e.g., 3 cycles). For example, analysis module 706 can compare the counter value to the cycle threshold. In response to determining that the counter value is equal to the cycle threshold, the analysis module 706 can output the endpoint notification. In some aspects, the analysis module 706 can delay outputting the endpoint notification for the threshold period.

Analysis module 706 can continue to monitor the negative component of the unipolar EGM. Analysis module 706 can output another notification to output module 708 if the negative component is detected (e.g., if the physician moves the catheter to another point for RF application). Analysis module 706 can reset the counter once the negative component is detected.

Output module 708 can be responsible for receiving the endpoint notification from analysis module 706. The endpoint notification may indicate to the physician that transmural lesion has been achieved and the physician can proceed to move the catheter to another point for RF application.

Output module 708 can label the signal data corresponding to the received endpoint notification. For example, in an embodiment where input source 702 is a catheter based signal and an alert corresponds to one of the catheters, output module 710 can change an attribute (e.g., the color, line thickness, background color, animation, or the like) on a unipolar EGM channel display to notify the physician of the endpoint notification. Output module 708 can forward the endpoint notification to a plurality of devices. In one embodiment, output module 708 can send the endpoint notification to display 710. In another embodiment, output module 708 can forward the endpoint notification to a device in electronic communication with system 700. Output module 708 can communicate and interact over a network with any combination of remote devices, remote networks, remote entities, etc. Each of these devices can be capable of receiving the endpoint notification. Moreover, in combination with the disclosed embodiments an audible alert can be generated by output module 708 when a given displayed signal's color is changed. Implementation of such an audible alert with hardware, firmware, software, and/or a combination thereof, would become apparent to persons of skill in the art.

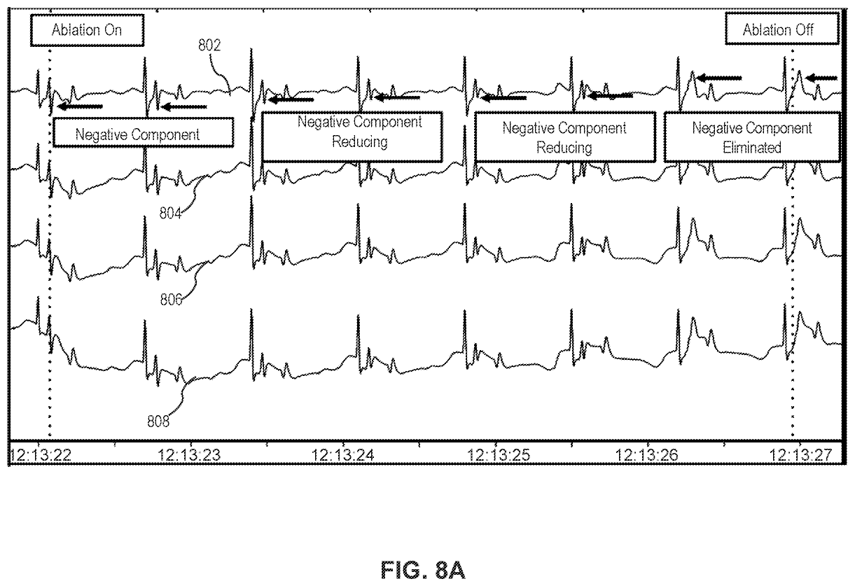

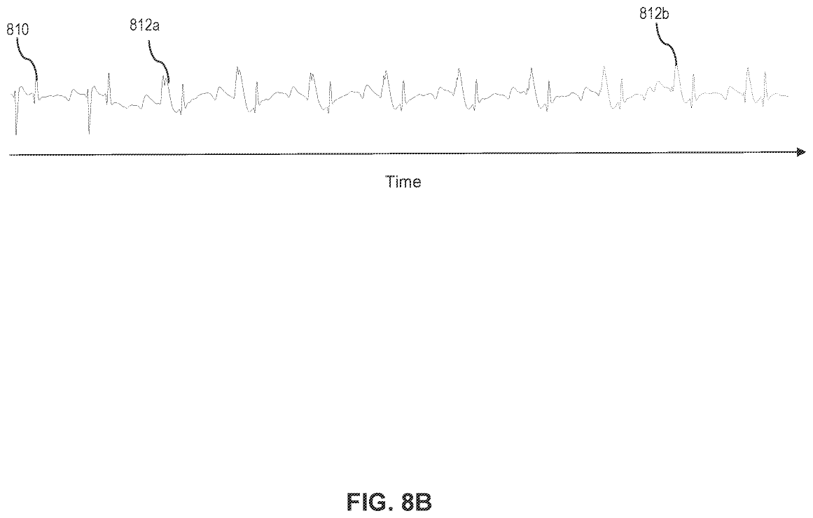

FIG. 8A in an example output of system 700 in accordance with some aspects of the present disclosure. The display can show one or more unipolar tracings at different filter settings. Each trace can correspond to a clean signal of the unipolar EGM data. FIG. 8A shows the unipolar tracings with four different filter settings. Trace 802 shows the unipolar tracing at 5 Hz, trace 804 shows the unipolar tracing at 1 Hz, trace 806 shows the unipolar tracing at 0.5 Hz, and trace 808 shows the unipolar tracing at 0.05 Hz. The filter settings may be predetermined and/or user adjusted different filtering settings. The two horizontal lines show the start of the ablation (e.g., RF turned on) and the end of the ablation (e.g., RF turned off). At the start of the ablation, the negative component is present in the unipolar EGM as shown in trace 802, trace 804, trace 806, and trace 808. After the negative component is eliminated, the ablation may be stopped.

FIG. 8B is an example output of system 700 in accordance with some aspects of the present disclosure. If the negative component of the EGM is present, unipolar EGM data 810 will be displayed in a first color 812a (the first color being represented by a solid line for illustration). Conversely, if the negative component has been eliminated for at least the threshold number of cycles (e.g., 3 cycles in FIG. 8B) then unipolar EGM data 810 can be displayed in a second color 812b (the second color being represented by a dashed line for illustration). As described previously herein this may be an indication for the physician that the RF application may be stopped.

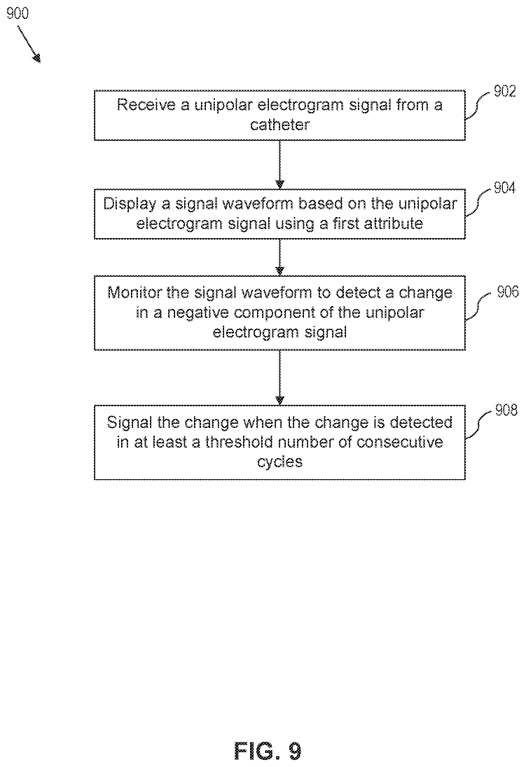

FIG. 9 is a flowchart for a method 900, in accordance with some aspects of the present disclosure. It is to be appreciated that not all steps can be needed to perform the disclosure provided herein. Further, some of the steps can be performed simultaneously, or in a different order than shown in FIG. 9, as will be understood by a person of ordinary skill in the art.

Method 900 can be implemented and performed by computing device 702. However, method 900 is not limited to that example aspect.

In 902, a unipolar electrogram signal from a catheter may be received. The unipolar electrogram signal can correspond to the electrical activity of a heart.

In 904, a signal waveform is displayed based on the unipolar electrogram signal using a first attribute. For example, the signal waveform may be displayed on a digital display using a first color. The signal waveform represents one or more cycles from the heart.

In 906, the signal waveform is monitored to detect a change in a negative component of the unipolar electrogram signal. A determination is made to whether the unipolar electrogram has the negative component. For example, a potential of the unipolar electrogram signal may be compared with the baseline. As described previously herein, the baseline may be determined before the RF application is started. A counter may be updated when the negative component is not detected in the unipolar electrogram signal. For example, the counter may be incremented by an incremental value (e.g., one) for each cycle where the negative is not detected. The counter is compared with the cycle threshold (threshold number of consecutive cycles).

In 908, the change is signaled on the at least one digital display when the change is detected in at least a threshold number of consecutive cycles. For example, after the change is detected for the threshold number of consecutive cycles, the signal waveform may be displayed on the digital display using a second attribute (e.g., a second color). In some aspects, the first attribute may be different from the second attribute. For example, the first attribute may be displaying the waveform using a blue color and the second attribute may be displaying the waveform using a red color. In some aspects, the signaling is delayed for the threshold period (e.g., 3 seconds). Thus, after the counter is equal to the threshold number of consecutive cycles, the waveform continues to be displayed using the first attribute until the threshold period has elapsed. After the threshold period has elapsed, the waveform is displayed using the second attribute. This indicates to the physician that the RF application may be stopped.

In some aspects, the signaling may include outputting an annotation on the digital display. For example, “Transmural lesion achieved” may be displayed on the digital display.

In some aspects, the signal waveform is continuously monitored. In response to detecting a negative component of the unipolar electrogram signal, the signal waveform is displayed using the first attribute (e.g., the display color is back to blue).

In some embodiments, an indication of the change is displayed at an application spot on a digital representation of the heart. The indication indicates that the therapy at the application spot is successfully completed. For example, a three dimensional representation of the heart may be output on the digital display. Once the change is detected (i.e., the negative component is eliminated for at least the predetermined threshold number of cycles), an indication of the elimination of the negative component at the application spot is output. For example, an attribute of the digital representation of the application spot is changed (e.g., color change). In some aspects, a text indicating that the treatment is completed at the position is output.

Another technological problem observed by the inventor was that during the setting of an EP study, the pattern of electrical activation within the patient's heart may change abruptly. This change can be visualized as a conduction pattern change on the surface ECG or static intracardiac catheters. Failure to promptly recognize this change can cause collection of inaccurate data and misdiagnosis. At the very least, failure to recognize the conduction pattern change can cause significant extension of procedure time. Often times, these changes are very subtle and can be missed by the naked eye. Prompt and accurate conduction pattern monitoring and reporting is an essential tool for this purpose.

One technological solution is that once the patient is in the rhythm of interest, a pattern of conduction can be saved by the monitoring system, such as PureEP™, manufactured by BioSig Technologies, Inc., of Westport, CT. A reference source can be selected. This reference may be a surface ECG signal, or signal from a catheter inside the heart. Two additional sample signal sources may be selected. The timing relationships between the two additional signal sources and the selected reference can be stored from the rhythm of interest. A user selected tolerance value in milliseconds may be programmed to allow for small vacillations in the real time measurements. This is appropriate for certain heart rhythms that do not have a consistent activation pattern. Once the reference channel, measurement channels, and tolerance levels are programmed, the algorithm can begin to monitor the activation pattern of the rhythm in real time. Should the timing relationships be between any/all measurement channels for three consecutive cycles, the channel(s) that failed the pattern can be highlighted as bold in the live screen. Should the measurements fall back within the tolerance for three consecutive cycles, the measured channels can return to their normal color and presentation. Five different rhythm patterns may be stored and recalled for comparison to the current rhythm at any time by the operator.

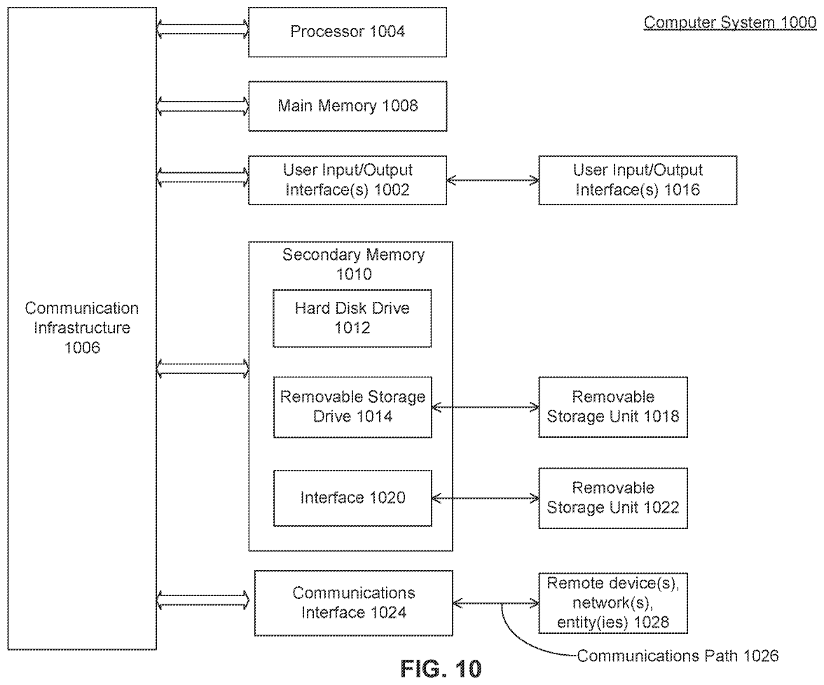

Various aspects can be implemented, for example, using one or more computer systems, such as computer system 1000 shown in FIG. 10. Computer system 1000 can be used, for example, to implement a system of monitoring cycle length changes and pattern changes within cardiac signal data. For example, computer system 1000 can receive one or more signals, display the signals, compare parameters of the signal to one or more templates, and display the signals in a different color if the signal parameters differ from the one or more templates. Computer system 1000 can be any computer capable of performing the functions described herein.

Computer system 1000 can be any well-known computer capable of performing the functions described herein.

Computer system 1000 includes one or more processors (also called central processing units, or CPUs), such as a processor 1004. Processor 1004 is connected to a communication infrastructure or bus 1006. Processor 1004 can be used to analyze incoming signals, access one or more templates, and compare values within the signals to the templates. Processor 1004 can also generate alerts if there are differences between the incoming signal values and the one or more templates.

One or more processors 1004 can each be a graphics processing unit (GPU). In an aspect, a GPU is a processor that is a specialized electronic circuit designed to process mathematically intensive applications. The GPU can have a parallel structure that is efficient for parallel processing of large blocks of data, such as mathematically intensive data common to computer graphics applications, images, videos, etc.

Computer system 1000 also includes user input/output device(s) 1016, such as a catheter, display/monitor(s), audio output device, keyboards, pointing devices, etc., that communicate with communication infrastructure 1006 through user input/output interface(s) 1002. Computer system 1000 can use input/output device(s) 1016 to receive one or more signals. Input/output device(s) 1016 can also be used to display the output of computer system 1000 on a monitor.

Computer system 1000 also includes a main or primary memory 1008, such as random access memory (RAM). Main memory 1008 can include one or more levels of cache. Main memory 1008 has stored therein control logic (i.e., computer software) and/or data.

Computer system 1000 can also include one or more secondary storage devices or memory 1010. Secondary memory 1010 can include, for example, a hard disk drive 1012 and/or a removable storage device or drive 1014. Removable storage drive 1014 can be a floppy disk drive, a magnetic tape drive, a compact disk drive, an optical storage device, tape backup device, and/or any other storage device/drive. Secondary memory 1010 can be used to record incoming signals for future analysis.

Removable storage drive 1014 can interact with a removable storage unit 1018. Removable storage unit 1018 includes a computer usable or readable storage device having stored thereon computer software (control logic) and/or data. Removable storage unit 1018 can be a solid state device, floppy disk, magnetic tape, compact disk, DVD, optical storage disk, and/any other computer data storage device. Removable storage drive 1014 reads from and/or writes to removable storage unit 1018 in a well-known manner.

According to an exemplary aspect, secondary memory 1010 can include other means, instrumentalities or other approaches for allowing computer programs and/or other instructions and/or data to be accessed by computer system 1000. Such means, instrumentalities or other approaches can include, for example, a removable storage unit 1022 and an interface 1020. Examples of the removable storage unit 1022 and the interface 1020 can include a program cartridge and cartridge interface (such as that found in video game devices), a removable solid state device or memory chip (such as an EPROM or PROM) and associated socket, a memory stick and USB port, a memory card and associated memory card slot, and/or any other removable storage unit and associated interface. Removable storage unit 1022 and interface 1020 can be used to input previously recorded signals to computer system 1000.

Computer system 1000 can further include a communication or network interface 1024. Communication interface 1024 enables computer system 1000 to communicate and interact with any combination of remote devices, remote networks, remote entities, etc. (individually and collectively referenced by reference number 1028). For example, communication interface 1024 can allow computer system 1000 to communicate with remote devices 1028 over communications path 1026, which can be wired and/or wireless, and which can include any combination of LANs, WANs, the Internet, etc. Control logic and/or data can be transmitted to and from computer system 1000 via communication path 1026.

In an aspect, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 1000, main memory 1008, secondary memory 1010, and removable storage units 1018 and 1022, as well as tangible articles of manufacture embodying any combination of the foregoing. Such control logic, when executed by one or more data processing devices (such as computer system 1000), causes such data processing devices to operate as described herein, such as for monitoring for cycle length changes, as described herein.

Based on the teachings contained in this disclosure, it will be apparent to persons skilled in the relevant art(s) how to make and use aspects of this disclosure using data processing devices, computer systems and/or computer architectures other than that shown in FIG. 10. In particular, aspects can operate with software, hardware, and/or operating system implementations other than those described herein.

What is claimed is:

1. A method for therapeutic signaling, the method comprising:
receiving, by at least one processor, a unipolar electrogram signal from a catheter;
displaying, on at least one digital display, a signal waveform based on the unipolar electrogram signal using a first attribute, wherein the signal waveform represents one or more cycles from a patient's heart;
monitoring, by the at least one processor, the signal waveform to detect a change in a negative component of the unipolar electrogram signal; and
signaling the change on the at least one digital display when the change is detected in at least a threshold number of consecutive cycles.

2. The method of claim 1, wherein the signaling further comprising:
displaying the signal waveform using a second attribute, the second attribute being different than the first attribute.

3. The method of claim 2, wherein the first attribute is displayed as a first color and the second attribute is displayed as a second color.

4. The method of claim 1, wherein the signaling further comprising:
displaying an annotation indicating the change on the signal waveform on the at least one digital display.

5. The method of claim 1, wherein the monitoring further comprising:
determining whether the unipolar electrogram has the negative component by comparing a potential of the unipolar electrogram signal to a baseline;
updating a counter when the negative component is not detected; and
comparing the counter to the at least threshold number of consecutive cycles.

6. The method of claim 5, further comprising:
dynamically identifying the baseline based on the one or more cycles received by the catheter.

7. The method of claim 1, wherein the threshold number comprises three cycles.

8. The method of claim 1, further comprising:
redisplaying, on the at least one digital display, the signal waveform using the first attribute in response to detecting the negative component of the unipolar electrogram signal.

9. The method of claim 1, further comprising:
delaying the signaling for a threshold period.

10. The method of claim 1, further comprising:
displaying a plurality of signal waveforms corresponding to a clean signal of the unipolar electrogram signal, wherein the plurality of signal waveforms are generated from the unipolar electrogram signal using predetermined and/or user adjusted different filtering settings; and
signaling the change on each signal waveform of the plurality of signal waveforms on the at least one digital display.

11. The method of claim 1, further comprising:
displaying an indication of the change at a location of a digital representation of the patient's heart, wherein the indication indicates that a therapy at the location is successfully completed.

12. A system, comprising:
a memory; and
at least one processor coupled to the memory and configured to:
receive a unipolar electrogram signal from a catheter;
display on at least one digital display a signal waveform based on the unipolar electrogram signal using a first attribute, wherein the signal waveform represents one or more cycles from a patient's heart;
monitor the signal waveform to detect a change in a negative component of the unipolar electrogram signal; and
signal the change on the at least one digital display when the change is detected in at least a threshold number of consecutive cycles.

13. The system of claim 12, wherein to signal the change, the at least one processor is further configured to:
display the signal waveform using a second attribute, the second attribute being different than the first attribute.

14. The system of claim 13, wherein the first attribute is displayed as a first color and the second attribute is displayed as a second color.

15. The system of claim 12, wherein to signal the change, the at least one processor is further configured to:
displaying an annotation indicating the change on the signal waveform on the at least one digital display.

16. The system of claim 12, wherein to monitor the signal waveform, the at least one processor is further configured to:
determine whether the unipolar electrogram has the negative component by comparing a potential of the unipolar electrogram signal to a baseline;
update a counter when the negative component is not detected; and
compare the counter to the at least threshold number of consecutive cycles.

17. The system of claim 16, wherein the processor is further configured to:
dynamically identify the baseline based on the one or more cycles received by the catheter.

18. The system of claim 12, wherein the threshold number comprises three cycles.

19. The system of claim 12, wherein the processor is further configured to:
redisplay, on the at least one digital display, the signal waveform using the first attribute in response to detecting the negative component of the unipolar electrogram signal.

20. The system of claim 12, wherein the processor is further configured to:
delay the signaling for a threshold period.

21. A non-transitory computer-readable device having instructions stored thereon that, when executed by at least one computing device, causes the at least one computing device to perform operations comprising:
receiving a unipolar electrogram signal from a catheter;
displaying, on at least one digital display, a signal waveform based on the unipolar electrogram signal using a first attribute, wherein the signal waveform represents one or more cycles from a patient's heart;
monitoring the signal waveform to detect a change in a negative component of the unipolar electrogram signal; and
signaling the change on the at least one digital display when the change is detected in at least a threshold number of consecutive cycles.
